(19) United States
(12) Patent Application Publication
(10) Pub. No.: US 2007/0038063 A1
(43) Pub. Date: Feb. 15, 2007

(54) Method for determining the position and orientation of an endoscopy capsule guided through an examination object by using a navigating magnetic field generated by means of a navigation device

(76) Inventors: Rainer Kuth, Herzogenaurach (DE); Johannes Reinschke, Nurnberg (DE); Rudolf Rockelein, Erlangen (DE)

Correspondence Address: HARNESS, DICKEY & PIERCE, P.L.C., P.O.BOX 8910, RESTON, VA, 20195, US

(21) Appl. No.: 11/481,935

(22) Filed: Jul. 7, 2006

(30) Foreign Application Priority Data
Jul. 8, 2005 (DE) 10 2005 032 370.7

(51) Int. Cl. A61B 5/05

(52) U.S. Cl. 600/407

(57) ABSTRACT

A method is disclosed for determining the position and orientation of an endoscopy capsule guided through an examination object by using a navigating magnetic field generated by way of a navigation device. In the method, an X-ray machine is used to record radiation images in which the endoscopy capsule is shown. Further, the position and orientation of the endoscopy capsule are determined with the aid of the position-dependent and orientation-dependent image of the endoscopy capsule in the radiation images.

### PRIORITY STATEMENT

The present application hereby claims priority under 35 U.S.C. §119 on German patent application number DE 10 2005 032 370.7 filed Jul. 8, 2005, the entire contents of which is hereby incorporated herein by reference.

### FIELD

The invention generally relates to a method for determining the position and orientation of an endoscopy capsule guided through an examination object by using a navigating magnetic field generated by way of a navigation device.

### BACKGROUND

In addition to the known catheter endoscopy, it has recently become known to use endoscopy capsules in order to record images or videos of the organ wall or the like, to take biopsies, set stents or the like in the interior of a hollow organ such as the gastrointestinal tract. To this end, the patient need only swallow a relatively small endoscopy capsule that migrates through the gastrointestinal tract and transmits the recorded image signals in a wireless fashion to an external control, operating or image processing device via an integrated image recording device and/or receives control signals from an external control or operating unit, etc. By comparison with conventional catheter endoscopy, examination and/or treatment by way of an endoscopy capsule is substantially more pleasant for the patient.

It is known to use a magnetic navigation device in order to be able to guide an endoscopy capsule actively. This navigation device generates in an extracorporeal fashion navigating magnetic fields that interact with at least one magnetic element integrated on the capsule side (for example permanent magnet, ferromagnetic material that can be magnetized in an external magnetic field, or electromagnetic coil). This renders it possible in a targeted fashion to exert a torque and/or a force on the capsule, to move the latter actively and to guide it through the gastrointestinal tract. Thus, there is no dependence on movement solely via the bowel peristalsis, but rather it is possible for guiding and movement to be active.

Navigation through the human body by way of these controlled magnetic navigating fields requires measurement of the location and the angle of the endoscopy capsule in absolute coordinates, that is to say relative to the navigating magnetic system, in order to be able to generate the correct magnetic fields and field gradients for the purpose of capsule navigation at the location of the capsule. There is therefore a need to determine the position and orientation of the capsule continuously.

Systems used to this end frequently operate with alternating magnetic fields. The patient is exposed to a variable inhomogeneous magnetic field. Currents are induced via this magnetic field in coil sensors on the capsule side. Respective location and/or orientation are/is determined by measuring the induced currents and from knowledge of the field distribution.

However, there is a problem here that magnetic fields are also generated during capsule navigation, and so there is the risk of these also interacting with the parts of the position acquiring system that are sensitive to magnetic fields, and of defective determination of position resulting in some cases.

There is thus the basic problem of specifying a method that enables position to be acquired without the navigating magnetic fields exerting an influence.

### SUMMARY

At least one embodiment of the invention provides that radiation images, in which the endoscopy capsule is shown, are recorded by using an X-ray machine. The position and orientation of the endoscopy capsule are determined with the aid of the position-dependent and orientation-dependent image of the endoscopy capsule in the radiation images.

At least one embodiment of the invention proposes the preferably intermittent recording of individual X-ray images in which, since the endoscopy capsule has a multiplicity of radiation-opaque elements, the endoscopy capsule is to be seen unambiguously as an image or shadow. Because of the known capsule geometry and/or the shape and geometry of the radiation-opaque elements in the capsule, these radiation images, which can be recorded in a clocked fashion with intervals from several 100 ms as far as the range of seconds, always exhibit an unambiguous image dependent on position and orientation. It is then possible straight away to conclude the position and orientation of the endoscopy capsule from an analysis of the image or the shadow image, this being done in all three spatial directions, as well as also with regard to the capsule rotation about its longitudinal axis, an endoscopy capsule generally being designed as an elongated cylinder, in order to enable the capsule to be easily swallowed or introduced, and also to enable ease of movement through the organs, which are usually constructed as elongated hollow organs.

Since the radiation-opaque capsule elements are usually metallic or are made from plastic doped with heavy atoms, or the like, they show a very clear image. Thus, it is possible to operate with an extremely low radiation dose, that is to say the radiation burden on the patient is minimal, since the capsule is imaged with an extremely high degree of contrast in the recorded radiation images.

Although the practiced user is capable straight away of detecting the coarse position and orientation directly with the aid of the radiation images displayed on a monitor, and of controlling the navigation device thereupon, an expedient development of at least one embodiment of the invention provides that the position data and orientation data are determined automatically via an image processing device and are transmitted to the navigation device and serve as the basis for subsequently controlling the generation of the magnetic field. In the course of this control of magnetic field generation, the several coils of the navigation device, for example 14 individually drivable coils, are driven separately as appropriate in order to be able at the location of the endoscopy capsule to generate the magnetic field which produces the desired force and/or torque. For example, the coils have the cross section of approximately 10 cm×10 cm, are filled with conductors by up to 70-80%, and are self-supporting. According to at least one embodiment of the invention, the coils are controlled as a function of the automatically acquired position and orientation data such that optimum field generation and thus movement control are possible.

As has been described, it is possible to record the radiation images intermittently in a consecutive fashion, that is to say to record and evaluate individual images successively in time, even several images per second being possible given that operating with extremely low radiation doses is possible.

In an advantageous embodiment, the system automatically increases the recorded images or the frequency of the measurements of location and solid angle when comparatively high fluxes are generated by the magnet system or have been generated in the previous seconds. Alternatively or in addition, the frequency is increased when the evaluation of location or solid angle ascertains a comparatively high difference from the previous measured value. That is to say, the image recording rate and/or the frequency of the measurements are/is varied as a function of the actual speed or the desired speed of the actively navigated capsule.

It is expedient, furthermore, when in order to determine the position and orientation, in addition to evaluating a currently recorded radiation image there is also an evaluation of one or more radiation images recorded earlier in time, such that the temporal movement path is also acquired over a specific period and is used in determining position and orientation. Of course, it is also possible thereby to perform a type of plausibility test, since consecutive position data and orientation data must be in a specific plausibility relationship to one another. Should this not be so, an error is present.

It is expedient, furthermore, when use is made of two positionally fixed X-ray sources that are at an angle to one another and have a common radiation detector via which radiation images are alternately recorded. Stereo principle is applied here because the two radiation sources are at an angle, but image on the common radiation detector. Upon exposure with a first X-ray source, an endoscopy capsule thus appears in another position and representation than upon exposure with the second radiation source. Consequently, a plausibility test can be performed, and it is also possible to resolve more effectively extreme positions such as, for example, positioning of the endoscopy capsule with its logical axis perpendicular to the detector plane.

At least one embodiment of the invention also relates to a medical examination device comprising an endoscopy capsule, a navigation device for generating a navigating magnetic field that interacts with at least one magnetic element provided in the endoscopy capsule, as well as at least one X-ray machine for recording radiation images in which the endoscopy capsule is shown, having an assigned image processing device for automatically determining position data and orientation data with the aid of the position-dependent and orientation-dependent image of the endoscopy capsule in the radiation images, which image processing device communicates with the navigation device in order to transmit the determined position data and orientation data. The endoscopy capsule may be, of course, connected to an assigned control device.

In the case of a design of the endoscopy capsule as an image recording capsule with an integrated image recording device (CCD camera or the like), the recorded image signals are transmitted in a wireless fashion to the control device in at least one embodiment, processed there and displayed on a monitor. It is also possible to use this control device to give control commands to the capsule, for example to switch on the image recording device, to drive a biopsy device that is integrated in the capsule, etc, depending on which functionalities are implemented at the endoscopy capsule.

According to at least one embodiment of the invention, the X-ray-opaque parts of the endoscopy capsule are shaped and/or arranged in such a way as to produce an asymmetric image in the case of an elongated endoscopy capsule whose longitudinal axis is perpendicular to the image recording plane of a radiation detector of the X-ray machine. It is thereby ensured that, even in the case of this extreme position in which a purely cylindrical image would be difficult to resolve with regard to the orientation and the solid angle, the capsule can also be resolved, because the orientation and the solid angle can be determined straight away via the asymmetry thereby obtaining. Conceivable here is, for example, an appropriate disk that has, for example, an indentation or the like and via which it is ensured that an appropriate asymmetric image is achieved.

The X-ray machine itself is expediently mechanically fastened on the tubular coil system generating the magnetic fields. As explained, the navigation device includes a multiplicity of, for example, 14 separate coils that form a tubular structure into which the patient is pushed while lying on a couch. The radiation source radiates through a coil opening, opposite which the solid state radiation detector is correspondingly arranged. That is to say, the X-ray machine with its two elements is therefore placed in or a little outside the coil system and can transradiate through corresponding coils. The X-ray detector (flat image detector) is always placed as close as possible to the volume (body) to be visualized, for example directly under the patient couch inside the coil system.

When the patient couch is swiveled inside the coil system (about its longitudinal axis), it can be sensible and requisite for the flat image detector also to be swiveled correspondingly. In this case, the X-ray source need not in any way be fixed, but can also be moved such that the X-ray source is always perpendicular above the flat image detector, that is to say the X-ray source and detector rotate jointly about the patient couch or the longitudinal axis of the coil system. It is also conceivable that only the X-ray source is mounted capable of being displaced mechanically along a circular movement in order to be able to emit “exposure flashes” from various “directions of view” relative to the detector.

It is expedient when two radiation sources arranged fixed in position and a common radiation detector are provided, the radiation sources being at an angle to one another and thus rendering stereo image recording operation possible. Given a fixed angle of the two sources to one another, the latter can also be mounted in a fashion capable of rotation about the axis of symmetry perpendicular to the detector.

### BRIEF DESCRIPTION OF THE DRAWINGS

Further advantages, features and details of the invention emerge from the example embodiment described below, as well as with the aid of the drawings, in which:

FIG. 1 shows a schematic of a medical examination device according to at least one embodiment of the invention,

FIG. 2 shows an enlarged schematic of an endoscopy capsule,

FIG. 3 shows a schematic for recording radiation images and for imaging the endoscopy capsule in the radiation image,

FIG. 4 shows a schematic with different capsule positions and the associated images,

FIG. 5 shows a plan view of an image in the case of an endoscopy capsule perpendicular to the detector plane, and

FIG. 6 shows a schematic with two X-ray sources.

### DETAILED DESCRIPTION OF THE EXAMPLE EMBODIMENTS

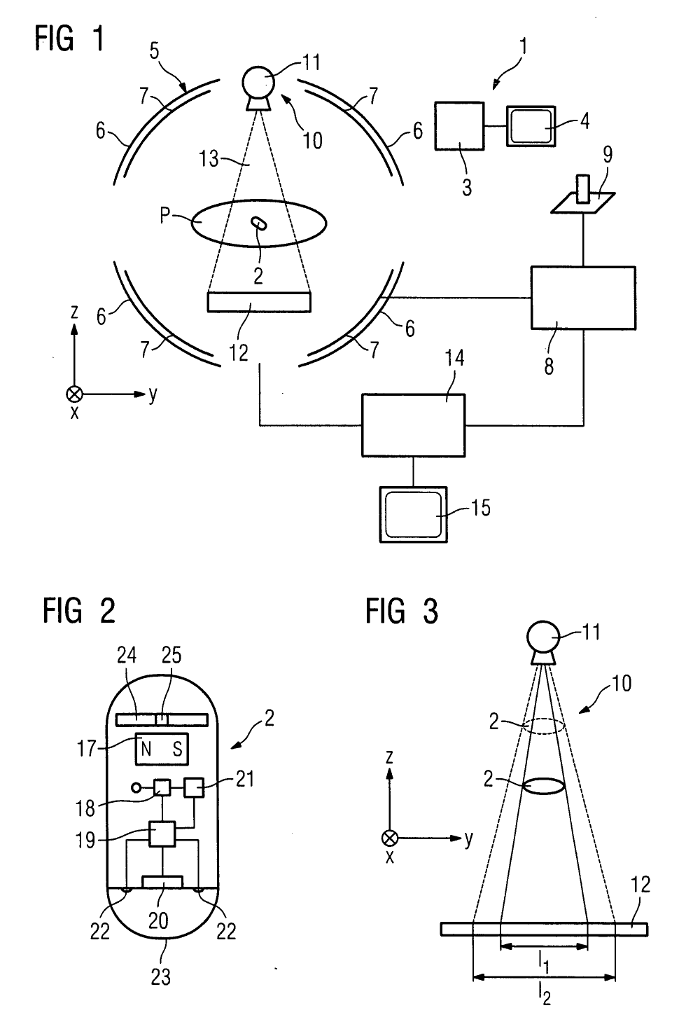

FIG. 1 shows a medical examination device 1 according to an embodiment of the invention that includes an endoscopy capsule 2 having an assigned control device 3 that communicates with it in a wireless fashion and via which all the functions of the endoscopy capsule 2 are controlled and, given design of the endoscopy capsule 2 as a video capsule or some other type of sensor capsule, to which the recorded image signals or measurement signals can also be transmitted and can be displayed on a monitor 4. In the example shown, the endoscopy capsule 2 is located in a patient P who has swallowed the capsule. The patient himself is located in a magnetic navigation device 5 via which the endoscopy capsule 2 can be moved actively through the patient.

The navigation device 5 in this case includes a number of separate coils 7 that form a coil system 6 and that can be driven or energized by a control device 8. As a rule, the several coils are known navigation devices that are arranged in a fashion distributed peripherally and axially and comprise approximately 14 separate coils. As set forth, the latter are all individually driven and, depending on position and orientation and the desired movement path of the endoscopy capsule, generate a magnetic field that acts appropriately at the capsule location and cooperates with a magnetic element in the interior of the endoscopy capsule 2, more detail being given thereon below. An operating element 9, for example a joystick that communicates with the control device 8, is provided for movement control. As an alternative to the manual input of the desired movement trajectory, it is also possible to provide an automatic desired trajectory determination.

Also arranged at the coil system 6 is an X-ray machine 10 including a radiation source 11 and, situated opposite, a solid state radiation detector 12, here a flat image detector. The X-ray source 11 and the detector 12 are arranged such that the emitted X-ray beam 13 can run through the coils 7 and be received without the coils 7 in any way impairing the image recording. The patient P and the endoscopy capsule 2 located in him are thereby detected, the endoscopy capsule being displayed, in the recorded radiation image, on a monitor 1S via a control device 14 that controls the operation of the X-ray machine 10 or is a part of the latter.

Furthermore, the control device 14 is capable of using the image of the endoscopy capsule 2 in the respectively recorded radiation image to determine the position data and solid angle data, that is to say the orientation of the endoscopy capsule 2 in the primary coordinate system, which is referred to the examination device itself or to the coil system 6, but not to the patient P. These determined position data and orientation data, which are given to the control device 8 in order to control the individual coils 7, can then be used to control an optimum magnetic field generation in order, via the generated magnetic fields, to exert on the endoscopy capsule 2 at the location thereof exactly those torques or forces that serve the purpose of the subsequent movement desired.

FIG. 2 shows an enlarged schematic of the endoscopy capsule 2. Firstly, it shows the magnetic element 17, which can be a permanent magnet, a magnetizable ferromagnetic element or an electromagnetic coil. Further, it shows a high-frequency transceiver 18 via which control signals that serve to operate the control device 19 on the capsule side, for example in the form of a processor, can be received, or signals can be transmitted by the control device 19, and also image signals that are recorded via the image recording device 20 can thereby be transmitted to the control device 3.

Also provided is a battery 21 for supplying power, as well as two illumination devices 22 in the form of small LEDs via which the hollow organ is illuminated such that the images can be recorded via the image recording device 20. The latter is encapsulated over an optically transparent window 23.

Also provided is a further X-ray-opaque element 24, many of the remaining elements described likewise being X-ray opaque, and thus forming a shadow or an image on a recorded radiation image. This element 24 is designed in the example shown as a disk that is circular and has an indentation 25, for example. This disk serves the purpose of imaging the capsule unambiguously when the capsule adopts an extreme position, that is to say when its longitudinal axis is perpendicular to the image recording plane of the radiation detector 12. It would be possible in this case for the orientation, that is to say the solid angle, to be determined only with difficulty. The integration of the disk-shaped element 24 with the unambiguous geometry has the effect, however, of producing an image as shown in FIG. 5. The image 26 shown there shows the circular outline of the disk-shaped element 24, as well as the corresponding indentation 27, resulting from the indentation 25, in the radiation image. It is thereby possible then to detect the solid angle orientation unambiguously in the coordinate system.

FIG. 3 shows the mode of operation of the X-ray machine 10 in the form of a schematic. It shows the X-ray source 11 together with the radiation detector 12, and the capsule 2 in two different positions. In the lower position (shown as a solid line), a somewhat smaller image is necessarily on the produced on the detector 12, indicated by l₁, since the endoscopy capsule 2 is positioned in the Z-direction nearer to the radiation detector 12. In the case of the endoscopy capsule 2 (shown as a dashed line) a substantially larger image results, indicated by l₂, since the endoscopy capsule 2 is spaced apart further from the detector 12 in the Z-direction. Of course, the capsule images are also correspondingly different when the endoscopy capsule is tilted about the X- and Y-axes or rotated in some other way in space. A position-specific and orientation-specific image or pattern is produced for each position and orientation as a consequence of the corresponding configuration of the X-ray-opaque elements in the capsule interior. The shape and geometry of the imaging elements are known to the control device 14, and so it can unambiguously analyze the images, and from that can determine the concrete X-, Y- and Z-coordinates as well as the solid angles with regard to the orientation of the endoscopy capsule solely from the image or shadow image.

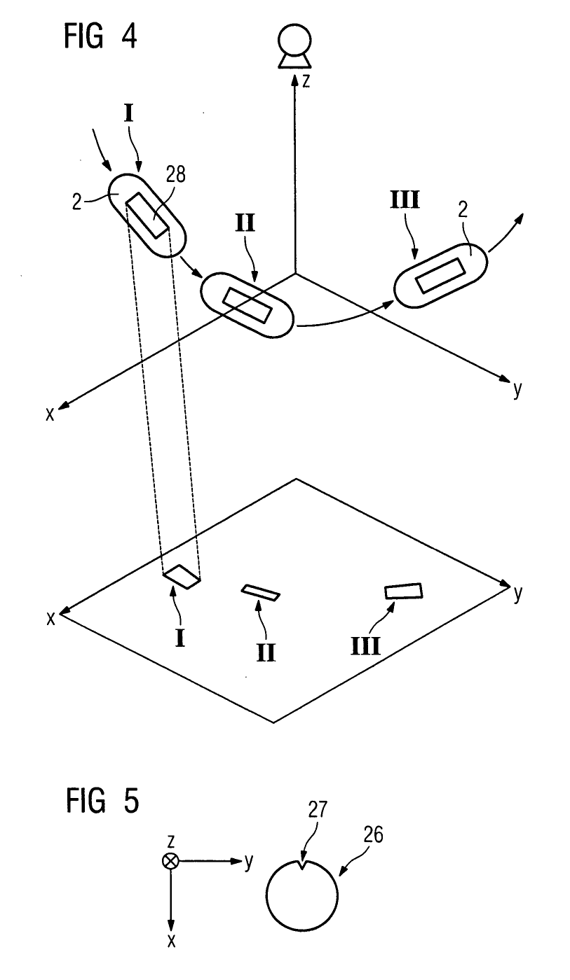

FIG. 4 shows a schematic of the endoscopy capsule 2 in various positions into which it is brought via the external navigation system. In position I, the capsule is oblique to the X-Y-plane and the rear end of the capsule is spaced apart in the Z-direction further from the detector plane, which lies in the X-Y-plane, than the front end of the capsule referred to the movement direction, which is illustrated by the arrows. Likewise illustrated in relation to the capsule 2, as a simplified cylinder, is the sum of the X-ray-opaque elements that are designated here overall by 28. If a radiation image is recorded, this leads to the graphic display designated again by I in the two-dimensional X-Y graphic display lying therebelow. Starting from the stylized cylinder 28, the graphic display is quadrangular, but asymmetric, the reason for this being that the cylinder 28 is, after all, at an angle to the X-Y-plane, and the front end is closer to the X-Y-plane than is the rear end. It therefore necessarily follows from recourse to the statements relating to FIG. 3 that the front end is imaged in a narrower fashion since it lies closer to the detector than does the rear end.

The position II, in which the capsule lies substantially horizontal in relation to the X-Y-plane, leads to a correspondingly rectangular image of the cylinder 28, as is designated by the image II.

An asymmetric image correspondingly results in turn when the endoscopy capsule is moved into the position III in which it is further removed from the detector plane with its leading end than with the rear end. This position is illustrated by III in the X-Y-plane in FIG. 4.

The control device 14 is now capable of using the respective representation of the image on the radiation detector for exact determination of the respective X-Y- and Z-coordinates and the solid angles. The position and geometry of the X-ray machine, that is to say the distance between the radiation source and radiation receiver, is fixed and known to the control device and so the corresponding parameters can be determined from the length or size of the image and from its shape.

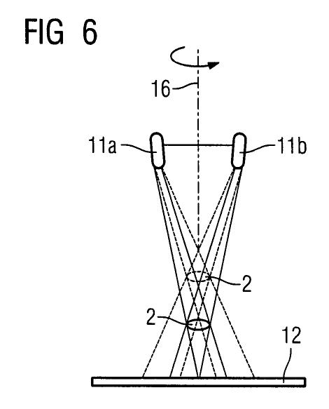

Finally, FIG. 6 shows a schematic of the mode of operation with two separate X-ray sources and a common detector. The two X-ray sources 11a, 11b are at an angle to one another and both radiate onto the common detector 12. It may be seen that the respective capsule 2, which is illustrated once in a lower position (with a solid line) and once in a higher position (with dots) depending on the radiation source 11a, 11b with which the radiation images are recorded, is illustrated respectively in a different position at the detector 12. Thus, it is possible to use these two radiation sources to operate with the aid of a stereotactic position measuring principle.

This stereotactic measuring principle becomes still more flexible when the radiation angle of the two sources is fixed, as the two sources are rotatably mounted as a whole where, the rotation axis 16 being the axis of symmetry between the two sources, which is perpendicular to the flat image detector 12, the two radiation sources being arranged on a common rotary holder.

Further, elements and/or features of different example embodiments may be combined with each other and/or substituted for each other within the scope of this disclosure and appended claims.

Still further, any one of the above-described and other example features of the present invention may be embodied in the form of an apparatus, method, system, computer program and computer program product. For example, of the aforementioned methods may be embodied in the form of a system or device, including, but not limited to, any of the structure for performing the methodology illustrated in the drawings.

Even further, any of the aforementioned methods may be embodied in the form of a program. The program may be stored on a computer readable media and is adapted to perform any one of the aforementioned methods when run on a computer device (a device including a processor). Thus, the storage medium or computer readable medium, is adapted to store information and is adapted to interact with a data processing facility or computer device to perform the method of any of the above mentioned embodiments.

The storage medium may be a built-in medium installed inside a computer device main body or a removable medium arranged so that it can be separated from the computer device main body. Examples of the built-in medium include, but are not limited to, rewriteable non-volatile memories, such as ROMs and flash memories, and hard disks. Examples of the removable medium include, but are not limited to, optical storage media such as CD-ROMs and DVDS; magneto-optical storage media, such as MOs; magnetism storage media, including but not limited to floppy disks (trademark), cassette tapes, and removable hard disks; media with a built-in rewriteable non-volatile memory, including but not limited to memory cards; and media with a built-in ROM, including but not limited to ROM cassettes; etc. Furthermore, various information regarding stored images, for example, property information, may be stored in any other form, or it may be provided in other ways.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A method for determining position and orientation of an endoscopy capsule guided through an examination object using a navigating magnetic field, the method comprising:
using an X-ray machine to record radiation images in which the endoscopy capsule is shown; and
determining the position and orientation of the endoscopy capsule with the aid of position-dependent and orientation-dependent image of the endoscopy capsule in the radiation images.

2. The method as claimed in claim 1, wherein the position data and orientation data are determined automatically via an image processing device and are transmitted to a navigation device, for generation of the navigating magnetic field, and serve as the basis for subsequently controlling the generation of the magnetic field.

3. The method as claimed in claim 1, wherein the radiation images are recorded intermittently.

4. The method as claimed in claim 3, wherein at least one of an image recording rate and a frequency of the position measurement and orientation measurement is varied as a function of at least one of an actual speed and a desired speed of the endoscopy capsule.

5. The method as claimed in claim 1, wherein at least one of
a radiation source and a radiation detector are fixed in position during image recording;
the radiation source is moved with reference to the positionally fixed radiation receiver; and
the radiation source and the radiation receiver rotate jointly about the longitudinal axis of the coil system of the navigation device while retaining their position relative to one another.

6. The method as claimed in claim 1, wherein, in order to determine the position and orientation, in addition to evaluating a currently recorded radiation image, there is also an evaluation of at least one radiation image recorded earlier in time.

7. The method as claimed in claim 1, wherein use is made of two X-ray sources that are at an angle to one another and have a common radiation detector via which radiation images are alternately recorded.

8. The method as claimed in claim 7, wherein at least one of
the two radiation sources are fixed in position during each recording; and
with reference to the positionally fixed radiation detector, the two radiation sources are moved about a rotation axis that is perpendicular to the image plane of the radiation detector.

9. A medical examination device, comprising:
an endoscopy capsule;
a navigation device to generate a navigating magnetic field that interacts with at least one magnetic element provided in the endoscopy capsule; and
at least one X-ray machine for recording radiation images in which the endoscopy capsule is shown, the at least one X-ray machine including an image processing device for automatically determining position data and orientation data with the aid of position-dependent and orientation-dependent imaging of the endoscopy capsule in the radiation images, the image processing device being further for communicating with the navigation device to transmit the determined position data and orientation data.

10. The examination device as claimed in claim 9, wherein X-ray opaque parts of the endoscopy capsule are at least one of shaped and arranged in such a way as to produce an asymmetric image in the case of an elongated endoscopy capsule whose longitudinal axis is perpendicular to the image recording plane of a radiation detector of the X-ray machine.

11. The examination device as claimed in claim 9, wherein the X-ray machine is mechanically fitted on the magnetic field generation coils forming a tubular coil system.

12. The examination device as claimed in claim 11, wherein at least one of
the radiation source and the radiation detector are fixed in position;
the radiation source is movable with reference to the positionally fixed radiation detector; and
the radiation source and the radiation detector are movable in common about the longitudinal axis of the coil system while retaining their position relative to one another.

13. The examination device as claimed in claim 9, wherein two radiation sources, positioned at an angle to one another, and a common radiation detector are provided.

14. The examination device as claimed in claim 13, wherein the two radiation sources are rotatable in common about a rotation axis perpendicular to the image plane of the radiation detector.

15. The method as claimed in claim 2, wherein the radiation images are recorded intermittently.

16. A method for determining position and orientation of an endoscopy capsule guided through an examination object using a navigating magnetic field, that the method comprising:
recording radiation images in which the endoscopy capsule is shown; and
determining the position and orientation of the endoscopy capsule with the aid of position-dependent and orientation-dependent image of the endoscopy capsule in the radiation images.

17. The method as claimed in claim 16, wherein the position data and orientation data are determined automatically via an image processing device and are transmitted to a navigation device, for generation of the navigating magnetic field, and serve as the basis for subsequently controlling the generation of the magnetic field.

18. The method as claimed in claim 16, wherein the radiation images are recorded intermittently.

19. The method as claimed in claim 17, wherein the radiation images are recorded intermittently.

20. The method as claimed in claim 16, wherein at least one of an image recording rate and a frequency of the position measurement and orientation measurement is varied as a function of at least one of an actual speed and a desired speed of the endoscopy capsule.

21. A medical examination device, comprising:
at least one X-ray machine for recording radiation images in which an endoscopy capsule is shown, the at least one X-ray machine including an image processing device for automatically determining position data and orientation data with the aid of position-dependent and orientation-dependent imaging of the endoscopy capsule in the radiation images, the image processing device being further for communicating with a navigation device for the endoscopy capsule, to transmit the determined position data and orientation data.

22. A medical device for determining position and orientation of an endoscopy capsule guided through an examination object using a navigating magnetic field, the medical device comprising:
means for recording radiation images in which the endoscopy capsule is shown; and
means for determining the position and orientation of the endoscopy capsule with the aid of position-dependent and orientation-dependent image of the endoscopy capsule in the radiation images.

23. A computer readable medium including program segments for, when executed on a computer, causing the computer to implement the method of claim 1.

24. A computer readable medium including program segments for, when executed on a computer, causing the computer to implement the method of claim 16.
